(19) United States
(12) Patent Application Publication
(10) Pub. No.: US 2014/0186411 A1
(43) Pub. Date: Jul. 3, 2014

(54) LOW VISCOSITY META-STABLE PHOTOPROTECTION COMPOSITION

(71) Applicant: L'OREAL, Paris (FR)

(72) Inventors: Anil SHAH, East Windsor, NJ (US); Jean-Thierry SIMONNET, Mamaroneck, NY (US); Carlos CRISSIEN, Clifton, NJ (US); Susan HALPERN, Basking Ridge, NJ (US); Kevin NIKITCZUK, North Brunswick, NJ (US)

(73) Assignee: L'OREAL, Paris (FR)

(21) Appl. No.: 13/727,739

(22) Filed: Dec. 27, 2012

(51) Int. Cl. A61K 8/06; A61Q 17/04

(52) U.S. Cl. CPC A61K 8/064; A61Q 17/04
USPC 424/401; 424/59

(57) ABSTRACT

A meta-stable photoprotective composition, a product including the meta-stable composition, and a method of protecting a keratinous substrate from UV radiation by applying the product are provided. The meta-stable photoprotective composition includes at least one water-in-oil emulsifier having a hydrophilic-lipophilic balance (HLB) of less than 6, at least one non-ionic oil-in-water emulsifier having an HLB of greater than 9, at least one ionic surfactant, and at least one sunscreen active ingredient. The weight ratio of the at least one non-ionic oil-in-water emulsifier to the at least one water-in-oil emulsifier is about 0.2 to about 2.0. The meta-stable composition converts from an oil-in-water (O/W) emulsion to a water-in-oil (W/O) emulsion upon rubbing.

### FIELD OF THE INVENTION

The present invention is directed to a meta-stable photoprotection composition, products, and method of using the meta-stable photoprotection composition. More specifically, the present invention is directed to a meta-stable photoprotection composition that converts from an oil-in-water (O/W) emulsion to a water-in-oil (W/O) emulsion upon rubbing.

### BACKGROUND OF THE INVENTION

Aging skin is the result of more than just chronological age. Skin is exposed to various environmental stresses, such as UV rays, which cause free radicals to form in the skin. Free radicals include, for example, singlet oxygen, hydroxyl radical, the superoxide anion, nitric oxide and hydrogen radicals. Free radicals attack DNA, membrane lipids and proteins, generating carbon radicals. These in turn react with oxygen to produce a peroxyl radical which may attack adjacent fatty acids to generate new carbon radicals. This process can lead to a chain reaction producing lipid peroxidation products. Damage to the cell membrane can result in loss of cell permeability, increased intercellular ionic concentration and/or decreased ability to excrete or detoxify waste products. The end result is a loss of elasticity of the skin and the appearance of wrinkles leading to premature aging of the skin. This process is commonly referred to as photo-aging.

Conventional sunscreen compositions are expected to possess water-resistance properties in order to inhibit the protective composition from being easily removed from a keratinous substrate by sweat and exposure to water. In order to achieve this function, film-formers are typically employed in the composition. By forming a film on the surface of keratinous substrates, the sunscreen actives are more steadfastly held in place upon exposure to water. The use of film-formers; however, has a negative impact of the tactile properties of the composition, making the composition feel tacky to the user.

As water-in-oil (W/O) emulsion systems are close to the skin's hydrolipid film, they are more effective from the dermatological viewpoint. This emulsion type promotes the long lasting moisturizing efficacy by providing an occlusive film and reinforces the active ingredients into the stratum corneum. In addition, W/O emulsions leave a lipophilic film on the skin surface which ensures high water repellency, which is an important parameter to maintain high UV protection in sun care applications. Nevertheless, tackiness, combined with greasiness and slow spreading are key factors which tend to decrease cosmetic acceptance, thus counteracting W/O emulsion benefits.

Therefore, it is desirable to provide a meta-stable composition that is applied as an oil-in-water (O/W) emulsion which quickly inverts to a water-in-oil emulsion (W/O) upon rubbing into the skin and that is pleasing to consumers.

A meta-stable photoprotection composition, product, and method of using the meta-stable photoprotection composition that do not suffer from one or more of the above drawbacks would be desirable in the art.

### BRIEF DESCRIPTION OF THE INVENTION

In an exemplary embodiment, a meta-stable photoprotection composition is provided. The meta-stable photoprotection composition includes at least one water-in-oil emulsifier having a hydrophilic-lipophilic balance (HLB) of less than 6, at least one non-ionic oil-in-water emulsifier having an HLB of greater than 9, at least one ionic surfactant; and at least one sunscreen active ingredient, wherein the ratio of the at least one non-ionic oil-in-water emulsifier to the at least one water-in-oil emulsifier is about 0.2 to about 2.0.

In another exemplary embodiment, a product formed from a meta-stable photoprotection composition is provided. The product includes at least one water-in-oil emulsifier having a hydrophilic-lipophilic balance (HLB) of less than 6, at least one non-ionic oil-in-water emulsifier having an HLB of greater than 9, at least one ionic surfactant, and at least one sunscreen active ingredient. The ratio of the at least one non-ionic oil-in-water emulsifier to the at least one water-in-oil emulsifier is about 0.2 to about 2.0.

The present disclosure is also directed to a method of protecting a keratinous substrate from UV radiation comprising applying onto a surface of the keratinous substrate a sunscreen product formed from a meta-stable photoprotection composition.

Other features and advantages of the present invention will be apparent from the following more detailed description of the preferred embodiment which illustrates, by way of example, the principles of the invention.

### DETAILED DESCRIPTION OF THE INVENTION

“Meta-stable composition,” as used herein, includes a composition that remains stable at least one month in storage at 45° C.

“W/O emulsion,” as used herein, includes a water phase dispersed in an oil phase, where the oil phase is a continuous phase.

“O/W emulsion,” as used herein, includes oil phase dispersed in a water phase, where the water phase is a continuous phase.

“Keratinous tissue,” as used herein, includes, but is not limited to, skin, hair, and nails.

“Homogenous” means substantially uniform throughout, i.e., a single phase mixture.

In the present application the term “ambient temperature” means a temperature of about 25° C.

The compositions and methods of the present invention can comprise, consist of, or consist essentially of the essential elements and limitations of the invention described herein, as well as any additional or optional ingredients, components, or limitations described herein or otherwise useful in personal care compositions intended for topical application to keratinous tissue.

It has been surprisingly discovered by the inventors that the compositions according to the present disclosure allow the formulation of stable and sprayable O/W emulsions, which invert very quickly into a W/O emulsion while rubbing on keratinous tissue, like the skin. Additionally, it has been surprisingly discovered by the inventors that composition has a great aesthetic profile like that of an O/W emulsion upon applying to the skin but has the final efficacy of a W/O emulsion.

W/O Emulsifiers

The oil phase present in the photoprotection composition, according to the disclosure, includes at least one water-in-oil (W/O) emulsifier at a concentration by weight of about 0.8% to about 2.8%, or alternatively about 0.9% to about 2.6%, based upon weight of the composition.

The at least one W/O emulsifier has an HLB of less than 6. Examples of W/O emulsifiers, include, but are not limited to, sucrose distearate, polyglyceryl-4 isostearate (and) cetyl PEG/PPG 10/1 dimethicone (and) hexyl laurate, sorbitan stearate, octyldodecyl xyloside, glyceryl stearate, and combinations thereof.

Typically, the W/O emulsifiers (component b) are chosen from the group consisting of sorbitan esters and polyglycerol esters:

Sorbitan esters: Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Additional products of 1 to 30 mol, and preferably 5 to 10 mol, ethylene oxide onto the sorbitan esters mentioned are also suitable.

Polyplycerol esters: Typical examples of suitable polyglycerol esters are Polyglyceryl-2 Dipolyhydroxystearate (Dehymuls® PGPH), Polyglycerin-3-Diisostearate (Lameform® TGI), Polyglyceryl-4 Isostearate (Isolan® GI 34), Polyglyceryl-3 Oleate, Diisostearoyl Polyglycerly-3 Diisostearate (Isolan® PDI), Polyglyceryl-3 Methylglucose Distearate (Tego Care® 450), Polyglyceryl-3 Beeswax (Cera Bellina®), Polyglyceryl-4 Caprate (Polyglycerol Caprate T2010/90), Polyglyceryl-3 Cetyl Ether (Chimexane® NL), Polyglyceryl-3 Distearate (Cremophor® GS 32) and Polyglyceryl Polyricinoleate (Admul®WOL 1403), Polyglyceryl Dimerate Isostearate and mixtues thereof. Examples of other suitable polyolesters are the mono-, di- and triesters of trimethylol propane or pentaerythritol with lauric acid, cocofatty acid, tallow fatty acid, palmitic acid, stearic acid, oleic acid, behenic acid and the like optionally reacted with 1 to 30 mol ethylene oxide.

Non-Ionic O/W Emulsifiers

The water phase in the meta-stable photoprotection composition, according to the disclosure, includes at least one non-ionic oil-in-water (O/W) emulsifier at a concentration by weight of about 0.2% to about 2.0%, or alternatively about 0.3% to about 1.9%, or alternatively about 0.4% to about 1.8%, based upon weight of the composition.

The at least on non-ionic O/W emulsifier has an HLB of greater than 9.

The non-ionic oil-in-water (O/W) emulsifier of the invention is preferably chosen from:

1) silicone surfactants,

2) amphiphilic lipids which are liquid at a temperature of less than or equal to 45° C. chosen from esters of at least one polyol and of at least one fatty acid including at least one saturated or unsaturated and linear or branched, and in particular unsaturated or branched, C₈-C₂₂ alkyl chain, the polyol being chosen from the group formed by polyethylene glycol including from 1 to 60 ethylene oxide units, sorbitan, glycerol possibly including from 2 to 30 ethylene oxide units, and polyglycerols including from 2 to 15 glycerol units,

3) esters of fatty acid and of sugar and ethers of fatty alcohol and of sugar,

4) surfactants which are solid at a temperature of less than or equal to 45° C. chosen from glycerol fatty esters, sorbitan fatty esters and oxyethylenated sorbitan fatty esters, ethoxylated fatty ethers and ethoxylated fatty esters,

5) block copolymers of ethylene oxide (A) and of propylene oxide (B), and the mixtures of these surfactants.

6) The silicone surfactants which can be used according to the invention are silicone compounds including at least one oxyethylene —OCH₂CH₂— chain and/or oxypropylene —OCH₂CH₂CH₂— chain. Mention may be made, as silicone surfactants which can be used according to the present invention, of those disclosed in documents U.S. Pat. No. 5,364,633 and U.S. Pat. No. 5,411,744, the entire contents of each of which are hereby incorporated by reference.

The silicone surfactant used according to the present invention is preferably a compound of formula (I):

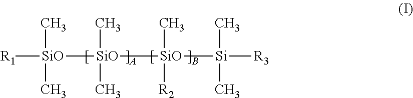

in which:

R₁, R₂ and R₃, independently of one another, represent a C₁-C₆ alkyl radical or a

—(CH₂)ₓ—(OCH₂CH₂)_{y}—(OCH₂CH₂CH₂)_{z}—OR₄ radical, at least one R₁, R₂ or R₃ radical not being an alkyl radical; R₄ being a hydrogen, an alkyl radical or an acyl radical;

A is an integer ranging from 0 to 200 (which range expressly includes 5, 20, 50, 100, and 150);

B is an integer ranging from 0 to 50 (which range expressly includes 5, 10, 25, 35, and 45); provided that A and B are not equal to zero at the same time;

x is an integer ranging from 1 to 6 (which range expressly includes 2, 3, 4, and 5);

y is an integer ranging from 1 to 30 (which range expressly includes 5, 10, 15, 20 and 25);

z is an integer ranging from 0 to 5 (which range expressly includes 1, 2, 3, and 4).

According to a preferred embodiment of the invention, in the compound of formula (I), the alkyl radical is a methyl radical, x is an integer ranging from 2 to 6 and y is an integer ranging from 4 to 30.

Mention may be made, as examples of silicone surfactants of formula (I), of the compounds of formula (II):

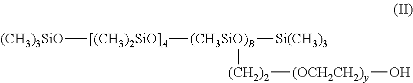

in which A is an integer ranging from 20 to 105 (which range expressly includes 30, 40, 50, 75 and 100), B is an integer ranging from 2 to 10 (which range expressly includes 3, 4, 5, 6, 7 and 8) and y is an integer ranging from 10 to 20 (which range expressly includes 12, 14, 16, and 18).

Mention may also be made, as examples of silicone surfactants of formula (I), of the compounds of formula (III):

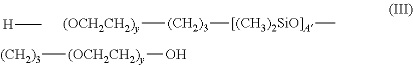

in which A′ and y are integers ranging from 10 to 20 (which range expressly includes 12, 14, 16, and 18).

Use may in particular be made, as silicone surfactants, of those sold by Dow Corning under the names DC 5329, DC 7439-146, DC 2-5695 and Q4-3667. The compounds DC 5329, DC 7439-146 and DC 2-5695 are compounds of formula (II) where respectively A is 22, B is 2 and y is 12; A is 103, B is 10 and y is 12; A is 27, B is 3 and y is 12.

2) The esters of fatty acid and of sugar can be chosen in particular from the group including esters or mixtures of esters of C₈-C₂₂ fatty acid and of sucrose, of maltose, of glucose or of fructose and esters or mixtures of esters of C₁₄-C₂₂ fatty acid and of methylglucose.

The C₈-C₂₂ or C₁₄-C₂₂ fatty acids forming the fatty unit of the esters which can be saturated or unsaturated linear alkyl chain having from 8 to 22 or from 14 to 22 carbon atoms respectively (which ranges expressly include 10, 12, 16, 18 and 20 as appropriate). The fatty unit of the esters can be chosen in particular from stearates, behenates, arachidonates, palmitates, myristates, laurates, caprates and their mixtures. Stearates are preferably used.

Mention may be made, as examples of esters or of mixtures of esters of fatty acid and of sucrose, of maltose, of glucose or of fructose, of sucrose monostearate, sucrose distearate, sucrose tristearate and their mixtures, such as the products sold by Croda under the name Crodesta F110 and F160 having respectively an HLB (Hydrophilic Lipophilic Balance) of 11 and 16; and, as examples of esters or of mixtures of esters of fatty acid and of methylglucose, of the distearate of methylglucose and of polyglycerol-3, sold by Goldschmidt under the name of Tego-care 450. Mention may also be made of glucose or maltose monoesters, such as methyl 0-hexadecanoyl6-D-glucoside and 0-hexadecanoyl-6-D-maltoside.

The ethers of fatty alcohol and of sugar which can be chosen in particular from the group including ethers or mixtures of ethers of C₈-C₂₂ fatty alcohol and of glucose, of maltose, of sucrose or of fructose and ethers or mixtures of ethers of C₁₄-C₂₂ fatty alcohol and of methylglucose. They are in particular alkylpolyglucosides.

The C₈-C₂₂ or C₁₄-C₂₂ fatty alcohols forming the fatty unit of the ethers which can be used in the nanoemulsion of the invention include a saturated or unsaturated linear alkyl chain having from 8 to 22 or from 14 to 22 carbon atoms respectively (which ranges expressly include 10, 12, 16, 18 and 20 as appropriate). The fatty unit of the ethers can be chosen in particular from the decyl, cetyl, behenyl, arachidyl, stearyl, palmityl, myristyl, lauryl, capryl or hexadecanoyl units and their mixtures, such as cetearyl.

Mention may be made, as examples of ethers of fatty alcohol and of sugar, of alkylpolyglucosides, such as decylglucoside and laurylglucoside, sold, for example, by Henkel under the respective names of Plantaren 2000 and Plantaren 1200, cetostearyl-glucoside, optionally as a mixture with cetostearyl alcohol, sold, for example, under the name Montanov 68 by Seppic, under the name Tego-care CG90 by Goldschmidt and under the name Emulgade KE3302 by Henkel, and arachidylglucoside, for example in the form of the mixture of arachidyl and behenyl alcohols and of arachidylglucoside sold under the name Montanov 202 by Seppic.

Use is more particularly made, as non-ionic amphiphilic lipid of this type, of sucrose monostearate, sucrose distearate, sucrose tristearate and their mixtures, the distearate of methylglucose and of polyglycerol-3, and alkylpolyglucosides.

The glycerol fatty esters can be chosen in particular from the group including the esters formed of at least one acid including a saturated linear alkyl chain having from 16 to 22 carbon atoms (which range expressly includes 18 and 20) and of 1 to 10 glycerol units (which range expressly includes 2, 3, 4, 5, 6, 7, 8 and 9). Use may be made of one or more of these glycerol fatty esters in the nanoemulsion of the invention.

The sorbitan fatty esters which can be chosen in particular from the group including esters of C₁₆-C₂₂ fatty acid and of sorbitan and oxyethylenated esters of C₁₆-C₂₂ fatty acid and of sorbitan. They are formed of at least one fatty acid including at least one saturated linear alkyl chain, having respectively from 16 to 22 carbon atoms, and of sorbitol or of ethoxylated sorbitol. The oxyethylenated esters generally include from 1 to 100 ethylene oxide units and preferably from 2 to 40 ethylene oxide (EO) units.

These esters can be chosen in particular from stearates, behenates, arachidates, palmitates and their mixtures.

The ethoxylated fatty ethers which are solid at a temperature of less than or equal to 45° C. which can be used as non-ionic amphiphilic lipids in the nanoemulsion according to the invention are preferably ethers formed of 1 to 100 ethylene oxide units and of at least one fatty alcohol chain having from 16 to 22 carbon atoms. The fatty chain of the ethers can be chosen in particular from the behenyl, arachidyl, stearyl or cetyl units and their mixtures, such as cetearyl. Mention may be made, as examples of ethoxylated fatty ethers, of the ethers of behenyl alcohol including 5, 10, 20 and 30 ethylene oxide units.

The ethoxylated fatty esters are esters formed of 1 to 100 ethylene oxide units and of at least one fatty acid chain including from 16 to 22 carbon atoms. The fatty chain of the esters can be chosen in particular from the stearate, behenate, arachidate or palmitate units and their mixtures.

3) The block copolymers of ethylene oxide and of propylene oxide, which can be used as non-ionic amphiphilic lipids in the nanoemulsion according to the invention can be chosen in particular from the block copolymers of formula (IV):

HO(C₂H₄O)ₓ(C₃H₆O)_{y}(C₂H₄O)_{z}H (IV)

in which x, y and z are integers such that x+z ranges from 2 to 100 (which range expressly includes 5, 10, 20, 40, 60, and 80) and y ranges from 14 to 60 (which range expressly includes 16, 18, 20, 30, 40 and 50), and their mixtures and more particularly from the block copolymers of formula (V) having an HLB ranging from 9 to 16 (which range expressly includes 4, 6, 8, 10, 12, and 14).

Ionic Surfactants

The water phase in the meta-stable composition, according to the disclosure, includes at least one ionic surfactant. The ionic surfactant may be an anionic surfactant or a cationic surfactant. The ionic surfactant is provided at a concentration by weight of about 0.1% to about 1.0%, or alternatively about 0.2% to about 0.9%, or alternatively about 0.3% to about 0.8%, based upon weight of the composition. The nature of the ionic surfactants is more important with respect to stability and the power to induce a spontaneous phase inversion of the emulsion when applied to skin.

Anionic surfactants useful as ionic surfactants in the present composition include anionic amphiphilic lipids which can be chosen from: 1) mixed esters of fatty acid or of fatty alcohol, of carboxylic acid and of glycerol, 2) alkyl ether citrates, 3) alkenyl succinates chosen from alkoxylated alkenyl succinates, alkoxylated glucose alkenyl succinates and alkoxylated methylglucose alkenyl succinates, and 4) phosphoric acid fatty esters.

Examples of the mixed esters of fatty acid or of fatty alcohol, of carboxylic acid and of glycerol which can be used as anionic amphiphilic lipids in the composition, according to the invention, can be chosen in particular from the group including mixed esters of fatty acid or of fatty alcohol having an alkyl chain including from 8 to 22 carbon atoms and of α-hydroxy acid and/or of succinic acid with glycerol. The α-hydroxy acid can be, for example, citric acid, lactic acid, glycolic acid, malic acid and their mixtures.

Examples of the alkyl chain of the fatty acids or alcohols from which the mixed esters which can be used as anionic surfactants in the composition of the invention derive can be saturated or unsaturated and linear or branched. It can, in particular, be stearate, isostearate, linoleate, oleate, behenate, arachidonate, palmitate, myristate, laurate, caprate, isostearyl, stearyl, linoleyl, oleyl, behenyl, myristyl, lauryl and capryl chains and their mixtures.

Mention may be made, as examples of mixed esters which can be used in the composition of the invention, of the mixed ester of glycerol and of the mixture of citric, lactic, linoleic and oleic acids (INCI name: Glyceryl citrate/lactate/linoleate/oleate) sold by Hills under the name Imwitor 375; the mixed ester of succinic acid and of isostearyl alcohol with glycerol (INCI name: Isostearyl diglyceryl succinate) sold by Huls under the name Imwitor 780 K; the mixed ester of citric acid and of stearic acid with glycerol (INCI name: Glyceryl stearate citrate) sold by Hills under the name Imwitor 370; or the mixed ester of lactic acid and of stearic acid with glycerol (INCI name: Glyceryl stearate lactate) sold by Danisco under the name Lactodan B30 or Rylo LA30.

Examples of the alkyl ether citrates, which can be used as anionic surfactants in the composition, according to the invention, can be chosen in particular from the group including the monoesters, diesters or triesters formed by citric acid and at least one oxyethylenated fatty alcohol, including a saturated or unsaturated and linear or branched alkyl chain having from 8 to 22 carbon atoms and including from 3 to 9 ethoxylated groups, and their mixtures. This is because it is possible to use a mixture of one or more of these citrates in the composition of the invention.

These citrates can be chosen, for example, from the mono-, di- and triesters of citric acid and of ethoxylated lauryl alcohol, including from 3 to 9 ethoxylated groups, sold by Witco under the name Witconol EC, in particular Witconol EC 2129, which is predominantly a dilaureth-9 citrate, and Witconol EC 3129, which is predominantly a trilaureth-9 citrate.

The alkyl ether citrates used as anionic surfactants lipids are preferably employed in the form neutralized to a pH of approximately 7, the neutralizing agent being chosen from inorganic bases, such as sodium hydroxide, potassium hydroxide or ammonia, and organic bases, such as mono-, di- and triethanolamine, aminomethylpropane-1,3-diol, N-methylglucamine or basic amino acids, such as arginine and lysine, and their mixtures.

The alkenyl succinates which can be used as anionic surfactants in the composition of the invention are in particular ethoxylated and/or propoxylated derivatives and they are preferably chosen from the compounds of formulae (V) or (VI):

HOOC—(HR)C—CH₂—COO-E (V)

HOOC—(HR)C—CH₂—COO-E-O—CO—CH₂—C(HR)—COOH (VI)

in which: the R and R′ radicals are chosen from linear or branched alkyl radicals including from 6 to 22 carbon atoms (which range expressly includes 10, 12, 14, 16, 18, and 20), E is chosen from oxyethylene chains of formula (C₂H₄O)ₙ in which n ranges from 2 to 100 (which range expressly includes 10, 20, 40, 60, 80 and 90), oxypropylene chains of formula (C₃H₆O)_{n'}, in which n′ ranges from 2 to 100 (which range expressly includes 5, 10, 20, 30, 40, 50, 60, 70, 80 and 90), random or blocked copolymers including 5 oxyethylene chains of formula (C₂H₄0)ₙ and oxypropylene chains of formula (C₃H₆O)_{n′} such that the sum of n and n′ ranges from 2 to 100 (which range expressly includes 5, 10, 20, 30, 40, 50, 60, 70, 80 and 90), oxyethylenated and/or oxypropylenated glucose groups including, on average, from 4 to 100 oxyethylene and/or oxypropylene units distributed over all the hydroxyl functional groups, or oxyethylenated and/or oxypropylenated methylglucose groups including, on average, from 4 to 100 oxyethylene and/or oxypropylene units distributed over all the hydroxyl functional groups (which ranges expressly include 5, 10, 20, 30, 40, 50, 60, 70, 80 and 90).

In the formulae (V) and (VI), n and n′ are mean values and are therefore not necessarily integers. The choice is advantageously made, for n, of a value ranging from 5 to 60 and more preferably still from 10 to 30.

The R and/or R′ radical is advantageously chosen from linear alkyl radicals including from 8 to 22 and preferably from 14 to 22 carbon atoms (which ranges expressly include 10, 12, 14, 16, 18 and 20 carbons as appropriate). Preferably, it can be, for example, the hexadecenyl radical, including 16 carbon atoms, or the octadecenyl radical, including 18 carbon atoms.

The compounds of formulae (V) and (VI) described above in which E is chosen from oxyethylene chains, oxypropylene chains and copolymers including oxyethylene chains and oxypropylene chains can be prepared in accordance with the description which is given in documents WO-A-94/00508, EP-A-1 071 99 and GB-A-2 131 820, the entire contents of each of which are incorporated herein by reference.

The acid functional group —COOH of the anionic surfactants of formulae (V) and (VI) is generally found in the composition of the invention in the form neutralized by a neutralizing agent, the neutralizing agent being chosen, for example, from inorganic bases, such as sodium hydroxide, potassium hydroxide or ammonia, and organic bases, such as mono-, di- and triethanolamine, aminomethylpropane-1,3-diol, N-methylglucamine or basic amino acids, such as arginine and lysine, and their mixtures.

Examples of the phosphoric acid fatty esters and their oxyethylenated derivatives, which can be used as anionic surfactants in the compositions, according to the invention, can be chosen in particular from the group including the esters formed of phosphoric acid and of at least one alcohol including a saturated or unsaturated and linear or branched alkyl chain having from 8 to 22 carbon atoms (which range expressly includes 10, 12, 14, 16, 18 and 20) and the esters formed of phosphoric acid and of at least one ethoxylated alcohol including a saturated or unsaturated and linear or branched alkyl chain having from 8 to 22 carbon atoms (which range expressly includes 10, 12, 14, 16, 18 and 20) and including from 2 to 40 oxyethylene groups (which range expressly includes 4, 6, 8, 10, 12, 14, 16, 18, 20 and 30), their salts and their mixtures. This is because it is possible to use a mixture of one or more of these phosphoric acid esters in the composition of the invention.

These esters can be chosen in particular from esters of phosphoric acid and of C₉-C₁₅ alcohols or their salts, such as the potassium salt of C₉-C₁₅ alkyl phosphate sold under the name Arlatone MAP by ICI; esters of phosphoric acid and of stearyl and/or isostearyl alcohols, such as the phosphate of stearyl/isostearyl alcohols (INCI name: Octyldecyl phosphate) sold under the name Hostaphat CG120 by Hoechst Celanese; esters of phosphoric acid and of cetyl alcohol, and their oxyethylenated derivatives, such as the product sold under the name Crodafos CES (mixture of cetearyl alcohol, of dicetyl phosphate and of ceteth-10 phosphate) by Croda; or esters of phosphoric acid and of tridecyl alcohol, and their oxyethylenated derivatives, such as the product sold under the name Crodafos T10 (INCI name: Trideceth-10 phosphate) by Croda. The oxyethylenated derivatives of phosphoric acid and of fatty alcohol can be prepared in accordance with the description given in Patent Application WO-A-96/14145, the entire contents of which is incorporated in the present application by reference.

Additional anionic surfactants which can be used in the compositions of the invention are preferably chosen from: alkaline salts of dicetyl and dimyristyl phosphate; alkaline salts of cholesterol sulfate; alkaline salts of cholesterol phosphate; lipoamino acids and their salts, such as mono- and disodium acylglutamates, for instance the disodium salt of N-stearoyl-L-glutamic acid sold under the name Acylglutamate HS21 by Ajinomoto; sodium salts of phosphatidic acid; phospholipids; alkylsulfonic derivatives, in particular of formula (VII):

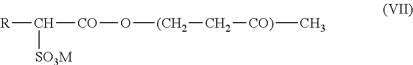

in which R represents C₁₆-C₂₂ alkyl radicals, in particular the C₁₆H₃₃ and C₁₈H₃₇ radicals taken as a mixture or separately, and M is an alkali metal or an alkaline earth metal, such as sodium; and their mixtures.

Examples of particularly suitable ionic surfactants, include, but are not limited to, ionic surfactant is chosen from sodium stearoyl glutamate, potassium cetyl phosphate, disodium stearoyl glutamate, and combinations thereof. The at least one ionic surfactant can be an anionic surfactant or cationic surfactant.

Cationic surfactants useful as ionic surfactants in the present composition include cationic amphiphilic lipids that are preferably chosen from the group formed by quaternary ammonium salts, fatty amines and salts thereof.

The quaternary ammonium salts are, for example: those which have the following general formula (VIII):

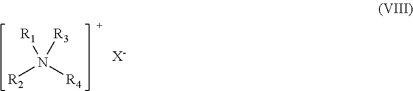

in which the radicals R₁ to R₄, which may be identical or different, represent a linear or branched aliphatic radical containing from 1 to 30 carbon atoms (which range expressly includes 2, 4, 6, 8, 10, 15, 20, and 25), or an aromatic radical such as aryl or alkylaryl. The aliphatic radicals may contain heteroatoms, such as in particular, oxygen, nitrogen, sulfur or halogens. The aliphatic radicals are, for example, chosen from the alkyl, alkoxy, polyoxy(C₂-C₆)alkylene, alkylamide, (C₁₂-C₂₂) alkylamido (C₂-C₆) alkyl, (C₁₂-C₂₂) alkyl acetate or hydroxyalkyl radicals containing from about 1 to 30 carbon atoms (which range expressly includes 2, 4, 6, 8, 10, 15, 20, and 25); X is an anion chosen from the group including halides, phosphates, acetates, lactates, (C₂-C₆) alkyl sulfates and alkyl- or alkylarylsulfonates, the quaternary ammonium salts of imidazolinium, such as, for example, that of the following formula (IX):

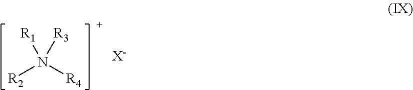

in which R₅ represents an alkenyl or alkyl radical containing from 8 to 30 carbon atoms (which range expressly includes 10, 12, 14, 16, 18, 20, 22 and 26) which are, for example, derived from tallow fatty acids, R₆ represents a hydrogen atom, a C₁-C₄ alkyl radical or an alkenyl or alkyl radical containing from 8 to 30 carbon atoms (which range expressly includes 10, 12, 14, 16, 18, 20, 22 and 26), R₇ represents a C₁-C₄ alkyl radical, R₈ represents a hydrogen atom, a C₁-C₄ alkyl radical, X is an anion chosen from the group including the halides, phosphates, acetates, lactates, alkyl sulfates, alkyl- or alkylarylsulfonates. Preferably, R₅ and R₆ designate a mixture of alkenyl or alkyl radicals containing from 12 to 21 carbon atoms which are, for example, derived from tallow fatty acids, R₇, designates methyl and R₈ designates hydrogen. Such a product is, for example, marketed under the name “REWOQUAT W 75” by the company REWO. Among the quaternary ammonium salts of formula (VIII), there are preferred, on the one hand, the tetraalkylammonium chlorides, such as, for example, the dialkyldimethylammonium or alkyltrimethylammonium chlorides, in which the alkyl radical contains from about 12 to 22 carbon atoms (which range expressly includes 14, 16, 18, and 22), in particular the behenyltrimethylammonium, distearyldimethylammonium, cetyltrimethylammonium and benzyldimethylstearyl-ammonium chlorides or alternatively, on the other hand, the stearamidopropyldimethyl(myristyl acetate)ammonium chloride marketed under the name “CERAPHYL 70” by the company VAN DYK. The behenyltrimethylammonium chloride is the quaternary ammonium salt most particularly preferred.

The quaternary diammonium salts of formula (X):

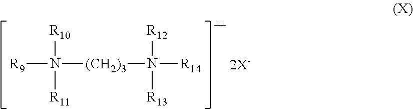

in which R₉ designates an aliphatic radical containing from about 16 to 30 carbon atoms, R₁₀, R₁₁, R₁₂, R₁₃ and the saturated or unsaturated, linear or branched C₁-C₆ hydrocarbon radicals R₂₂, the hydrogen atom, R₁₇, R₁₉ and R₂₁, which are identical or different, are chosen from the saturated or unsaturated, linear or branched C₇-C₂₁ hydrocarbon radicals; n, p and r, which are identical or different, are integers having values from 2 to 6 (which range expressly includes 3, 4, and 5); y is an integer having a value from 1 to 10 (which range expressly includes 2, 3, 4, 5, 6, 7, 8, and 9); x and z, which are identical or different, are integers having values from 0 to 10 (which range expressly includes 1, 2, 3, 4, 5, 6, 7, 8, and 9); X⁻ is an organic or inorganic, simple or complex anion (such as those described herein above and below); with the proviso that the sum x+y+z has a value from 1 to 15, that when x has a value of 0, then R₁₆ designates R₂₀, and that when z has a value of 0, then R₁₈ designates R₂₂. The alkyl radicals R₁₅ may be linear or branched and more particularly linear. Preferably, R₁₅ designates a methyl, ethyl, hydroxyethyl or dihydroxypropyl radical and more particularly a methyl or ethyl radical. Advantageously, the sum x+y+z has a value from 1 to 10 (which range expressly includes 2, 3, 4, 5, 6, 7, 8 and 9). When R₁₆ is a hydrocarbon radical R₂₀, it may be long and may have from 12 to 22 carbon atoms (which range expressly includes 14, 16, 18, and 20), or may be short and may have from 1, 2, or 3 carbon atoms. R₁₄, which are identical or different, are chosen from hydrogen or an alkyl radical containing from 1, 2, 3, or 4 carbon atoms, and X is an anion chosen from the group including the halides, acetates, phosphates, nitrates and methyl sulfates. Such quaternary diammonium salts include, in particular, propanetallowdiammonium dichloride.

The quaternary ammonium salts containing at least one ester functional group which can be used, according to the invention, are, for example, those of the following formula (XI):

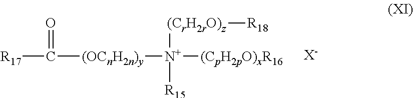

in which: R₁₅ is chosen from C₁-C₆ alkyl radicals and C₁-C₆ hydroxyalkyl or dihydroxyalkyl radicals; R₁₆ is chosen from:
the radical

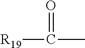

the saturated or unsaturated, linear or branched C₁-C₂₂ hydrocarbon radicals R₂₀, the hydrogen atom, R₁₈ is chosen from:
the radical

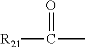

the saturated or unsaturated, linear or branched C₁-C₆ hydrocarbon radicals R₂₂, the hydrogen atom, R₁₇, R₁₉ and R₂₁, which are identical or different, are chosen from the saturated or unsaturated, linear or branched C₇-C₂₁ hydrocarbon radicals; n, p and r, which are identical or different, are integers having values from 2, 4, 5, or 6; y is an integer having a value from 1 to 10 (which range expressly includes 2, 3, 4, 5, 6, 7, 8, and 9); x and z, which are identical or different, are integers having values from 0 to 10 (which range expressly includes 1, 2, 3, 4, 5, 6, 7, 8, and 9); X⁻ is an organic or inorganic, simple or complex anion (such as those described hereinabove and below); with the proviso that the sum x+y+z has a value from 1 to 15, that when x has a value of 0, then R₁₆ designates R₂₀, and that when z has a value of 0, then R₁₈ designates R₂₂. The alkyl radicals R₁₅ may be linear or branched and more particularly linear. Preferably, R₁₅ designates a methyl, ethyl, hydroxyethyl or dihydroxypropyl radical and more particularly a methyl or ethyl radical. Advantageously, the sum x+y+z has a value from 1 to about 10. When R₁₆ is a hydrocarbon radical R₂₀, it may be long and may have from 12 to 22 carbon atoms (which range expressly includes 14, 16, 18 and 20), or may be short and may have from 1, 2, or 3 carbon atoms. When R₁₈ is a hydrocarbon radical R₂₂, it preferably has 1 to 3 carbon atoms. Advantageously, R₁₇, R₁, R₉ and R_{.21}, which are identical or different, are chosen from saturated or unsaturated, linear or branched C₁₁-C_{.21} hydrocarbon radicals, and more particularly from saturated or unsaturated, linear or branched C₁₁-C₂₁ alkyl and alkenyl radicals. Preferably, x and z, which are identical or different, have a value of 0 or 1. Advantageously, y is equal to 1. Preferably, n, p and r, which are identical or different, have a value of 2 or 3 and, still more particularly, are equal to 2.

In formula (XI), the anion is preferably a halide (chloride, bromide or iodide) or an alkyl sulfate, more particularly methyl sulfate. It is, however, possible to use methanesulfonate, phosphate, nitrate, tosylate, an organic acid-derived anion, such as acetate or lactate or any other anion compatible with ammonium containing an ester functional group. The anion X is still more particularly chloride or methyl sulfate.

Use is more particularly made of the ammonium salts of formula (XI) in which: R₁₅ designates a methyl or ethyl radical, x and y are equal to 1; z is equal to 0 or 1; n, p and r are equal to 2; R₁₆ is chosen from: the radical

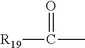

the methyl, ethyl or C₁₄-C₂₂ hydrocarbon radicals; the hydrogen atom R₁₈ is chosen from: the radical

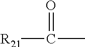

the hydrogen atom; R₁₇, R₁₉ and R₂₁, which are identical or different, are chosen from saturated or unsaturated, linear or branched C₁₃-C₁₇ hydrocarbon radicals and preferably from saturated or unsaturated, linear or branched C₁₃-C₁₇ alkyl and alkenyl radicals. Advantageously, the hydrocarbon radicals are linear.

There may be mentioned, for example, as compounds of formula (XI), the salts (chloride or methyl sulfate in particular) of diacyloxyethyldimethylammonium, of diacyloxyethyl-hydroxyethylmethylammonium, of monoacyloxyethyl-dihydroxyethylmethylammonium, of triacyloxyethylmethylammonium, of monoacyloxyethylhydroxyethyldimethylammonium and mixtures thereof. The acyl radicals preferably have 14 to 18 carbon atoms and are more particularly obtained from a vegetable oil, such as palm oil or sunflower oil. When the compound contains several acyl radicals, the latter may be identical or different. These products are obtained, for example, by direct esterification of triethanolamine, of triisopropanolamine, of alkyldiethanolamine or of alkyldiisopropanolamine which are optionally oxyalkylenated on fatty acids or on mixtures of fatty acids of plant or animal origin or by transesterification of methyl esters thereof. This esterification is followed by quaternization with the aid of an alkylating agent, such as an alkyl (preferably methyl or ethyl)halide, a dialkyl (preferably methyl or ethyl)sulfate, methyl methanesulfonate, methyl para-toluenesulfonate, or glycol or glycerol chlorohydrin. Such compounds are, for example, marketed under the names DEHYQUART by the company HENKEL, STEPANQUAT by the company STEPAN, NOXAMIUM by the company CECA, REWOQUAT WE 18 by the company REWO-WITCO.

The composition, according to the invention, when it contains ammonium salts, preferably contains a mixture of quaternary ammonium mono-, di- and triester salts, with a majority by weight of diester salts.

As a mixture of ammonium salts, there may be used, for example, the mixture containing 15% to 30% by weight of acyloxyethyldihydroxyethylmethylammonium methyl sulfate, 45% to 60% of diacyloxyethyl-hydroxyethylmethylammonium methyl sulfate and 15% to 30% of triacyloxyethylmethylammonium methyl sulfate, acyl radicals having from 14 to 18 carbon atoms and being obtained from optionally partially hydrogenated palm oil.

It is also possible to use the ammonium salts containing at least one ester functional group which are described in patents, U.S. Pat. No. 4,874,554 and U.S. Pat. No. 4,137,180, the entire contents of each of which are hereby incorporated by reference.

The combined concentration by weight of the at least one water-in-oil emulsifier, the at least one non-ionic oil-in-water emulsifier and the at least one ionic surfactant does not exceed about 4% by weight, based on the total weight of the composition.

The composition includes an oil phase containing surfactants. The surfactants in the oil phase comprise less than about 30% by weight of the oil phase, less than 20%, less than about 15% by weight of the oil phase, or alternatively less than about 10% by weight of the oil phase.

Sunscreen Active Ingredient

The aqueous phase or the oil phase, depending on the nature of the active ingredient, includes a sunscreen active ingredient to provide the composition with photoprotective properties. The meta-stable photoprotection composition, according to the disclosure, includes a sunscreen active ingredient at a concentration, by weight, of about 1% to about 40%, or alternatively about 1.5% to about 35%, or alternatively about 2% to about 30%, based upon weight of the photoprotection composition.

Suitable examples of sunscreen active ingredients include, but are not limited to, sunscreen actives, capryloyl salicylic acid, adenosine, baicalin, resveratrol, other polyphenols, free-radical scavengers, keratolytic agents, vitamins (e.g., Vitamin E and derivatives thereof), anti-elastase and anti-collagenase agents, peptides, fatty acid derivatives, steroids, trace elements, extracts of algae and of planktons, enzymes and coenzymes, flavonoids and ceramides, hydroxy acids and mixtures thereof, and enhancing agents. These active ingredients may be soluble or dispersible in whatever phase or phases is/are present in the meta-stable photoprotection composition (i.e., aqueous and/or fatty (oil) phase).

Examples of sunscreen active ingredients include, but are not limited to, butyl methoxydibenzoylmethane; anthranilates; salicylic derivatives; camphor derivatives; benzophenone derivatives; β,β-diphenylacrylate derivatives; triazine derivatives; benzotriazole derivatives; benzalmalonate derivatives; benzimidazole derivatives; imidazolines; bis-benzazolyl derivatives as described in patents EP 669 323 and U.S. Pat. No. 2,463,264; p-aminobenzoic acid (PABA) derivatives; methylenebis(hydroxyphenylbenzotriazole) derivatives as described in patent applications U.S. Pat. No. 5,237,071, U.S. Pat. No. 5,166,355, GB 2 303 549, DE 197 26 184 and EP 893 119; benzoxazole derivatives, such as those described in patent applications EP 0 832 642; EP 1 027 883, EP 1 300 137 and DE 101 62 844; screening polymers and screening silicones such as those described especially in patent application WO 93/04665; dimers derived from α-alkylstyrene, such as those described in patent application DE 198 55 649; 4,4-diarylbutadienes, such as those described in patent applications EP 0 967 200, DE 197 46 654, DE 197 55 649, EP-A-1 008 586, EP 1 133 980 and EP 133 981, and mixtures thereof, the entire contents of the patents and patent applications being incorporated by reference.

An example of an organic photoprotective agent as the sunscreen active ingredient, includes, but is not limited to, butyl methoxydibenzoylmethane sold by A&E Connock (Perfumery & Cosmetics) LTD., also known as Avobenzone and also sold under the names “Eusolex 9020” sold by Merck KGaA/EMD Chemicals Inc., “Neo Heliopan” sold by Symrise, “Parsol 1789” sold by DSM Nutritional Products and “Oristar ABZ” sold by Orient Stars LLC.

Examples of para-aminobenzoic acid derivatives, include, but are not limited to, PABA, ethyl PABA, ethyl dihydroxypropyl PABA, ethylhexyl dimethyl PABA sold in particular under the name “Escalol 507” by ISP, glyceryl PABA, and PEG-25 PABA sold under the name “Uvinul P25” by BASF.

Examples of salicylic derivatives, include, but are not limited to, homosalate sold under the name “Eusolex HMS” by Rona/EM Industries, ethylhexyl salicylate sold under the name “Neo Heliopan OS” by Haarmann and Reimer, dipropylene glycol salicylate sold under the name “Dipsal” by Scher, and TEA salicylate sold under the name “Neo Heliopan TS” by Haarmann and Reimer.

Examples of β,β-diphenylacrylate derivatives, include, but are not limited to, octocrylene sold, in particular, under the trade name “Uvinul N539” by BASF, and etocrylene sold, in particular, under the trade name “Uvinul N35” by BASF.

Examples of benzophenone derivatives, include, but are not limited to, benzophenone-1 sold under the trade name “Uvinul 400” by BASF, benzophenone-2 sold under the trade name “Uvinul D50” by BASF, benzophenone-3 or Oxybenzone sold under the trade name “Uvinul M40” by BASF, benzophenone-4 sold under the trade name “Uvinul MS40” by BASF, benzophenone-5, benzophenone-6 sold under the trade name “Helisorb 11” by Norquay, benzophenone-8 sold under the trade name “Spectra-Sorb UV-24” by American Cyanamid, benzophenone-9 sold under the trade name “Uvinul DS-49” by BASF, and benzophenone-12 diethylaminohydroxybenzoylhexyl benzoate sold under the tradename “Uvinul A Plus” by BASF.

Examples of benzylidenecamphor derivatives, include, but are not limited to, 3-benzylidenecamphor manufactured under the name “Mexoryl SD” by Chimex, 4-methylbenzylidenecamphor sold under the name “Eusolex 6300” by Merck, benzylidenecamphorsulfonic acid manufactured under the name “Mexoryl SL” by Chimex, camphor benzalkonium methosulfate manufactured under the name “Mexoryl SO” by Chimex, terephthalylidenedicamphorsulfonic acid manufactured under the name “Mexoryl SX” by Chimex, and polyacrylamidomethylbenzylidenecamphor manufactured under the name “Mexoryl SW” by Chimex.

Examples of phenylbenzimidazole derivatives, include, but are not limited to, phenylbenzimidazolesulfonic acid sold, in particular, under the trade name “Eusolex 232” by Merck, and disodium phenyl dibenzimidazole tetrasulfonate sold under the trade name “Neo Heliopan AP” by Haarmann and Reimer.

Examples of phenylbenzotriazole derivatives, include, but are not limited to, mention drometrizole trisiloxane sold under the name “Silatrizole” by Rhodia Chimie, and methylenebis(benzotriazolyl)tetramethylbutylphenol sold in solid form under the trade name “MIXXIM BB/100” by Fairmount Chemical, or in micronized form as an aqueous dispersion under the trade name “Tinosorb M” by Ciba Specialty Chemicals.

Examples of triazine derivatives, include, but are not limited to, bis(ethylhexyloxyphenol)methoxyphenyl triazine sold under the trade name “Tinosorb S” by Ciba-Geigy, Ethylhexyltriazone sold in particular under the trade name “Uvinul T150” by BASF, and diethylhexylbutamidotriazone sold under the trade name “Uvasorb HEB” by Sigma 3V, 2,4,6-tris(diisobutyl 4′-aminobenzalmalonate)-s-triazine.

An example of an anthranilic derivative, includes, but is not limited to, menthyl anthranilate sold under the trade name “Neo Heliopan MA” by Haarmann and Reimer.

An example of an imidazoline derivative, includes, but is not limited to, ethylhexyldimethoxybenzylidenedioxoimidazoline propionate.

An example of a benzalmalonate derivative, includes, but is not limited to, polyorganosiloxane containing benzalmalonate functions, for instance, Polysilicone-15, sold under the trade name “Parsol SLX” by Hoffmann LaRoche.

An example of a 4,4-diarylbutadiene derivative, includes, but is not limited to, 1,1-Dicarboxy(2,2′-dimethylpropyl)-4,4-diphenyl-butadiene.

An example of a benzoxazole derivative, includes, but is not limited to, 2,4-bis[5-(1-dimethylpropyl)benzoxazol-2-yl(4-phenyl)imino]-6-(2-ethylhex-yl)imino-1,3,5-triazine sold under the name Uvasorb K2A by Sigma 3V.

Examples of mineral sunscreen active ingredients are chosen from pigments and even more preferably nanopigments (mean size of the primary particles: generally between 5 nm and 100 nm and preferably between 10 nm and 50 nm) of treated or untreated metal oxides such as, for example, nanopigments of titanium oxide (amorphous or crystallized in rutile and/or anatase form), of iron oxide, of zinc oxide, of zirconium oxide or of cerium oxide.

The treated nanopigments are pigments that have undergone one or more surface treatments of chemical, electronic, mechanochemical and/or mechanical nature with compounds as described, for example, in Cosmetics & Toiletries, February 1990, Vol. 105, pp. 53-64, such as amino acids, beeswax, fatty acids, fatty alcohols, anionic surfactants, lecithins, sodium, potassium, zinc, iron or aluminum salts of fatty acids, metal (titanium or aluminum) alkoxides, poly-ethylene, silicones, proteins (collagen or elastin), alkanolamines, silicon oxides, metal oxides, sodium hexametaphosphate, alumina or glycerol.

The treated nanopigments may more particularly be titanium oxides treated with: silica and alumina, such as the products “Microtitanium Dioxide MT 500 SA” and “Microtitanium Dioxide MT 100 SA” from the company Tayca, and the products “Tioveil Fin”, “Tioveil OP”, “Tioveil MOTG” and “Tioveil IPM” from the company Tioxide; alumina and aluminum stearate, such as the product “Microtitanium Dioxide MT 100 T” from the company Tayca; alumina and aluminum laurate, such as the product “Microtitanium Dioxide MT 100 S” from the company Tayca; iron oxides and iron stearate, such as the product “Microtitanium Dioxide MT 100 F” from the company Tayca; silica, alumina and silicone, such as the products “Microtitanium Dioxide MT 100 SAS”, “Microtitanium Dioxide MT 600 SAS” and “Microtitanium Dioxide MT 500 SAS” from the company Tayca; sodium hexametaphosphate, such as the product “Microtitanium Dioxide MT 150 W” from the company Tayca; octyltrimethoxysilane, such as the product “T-805” from the company Degussa; alumina and stearic acid, such as the product “UVT-M160” from the company Kemira; alumina and glycerol, such as the product “UVT-M212” from the company Kemira; alumina and silicone, such as the product “UVT-M262” from the company Kemira.

Other titanium oxide nanopigments treated with a silicone are preferably TiO₂ treated with octyltrimethylsilane and for which the mean size of the elementary particles is between 25 and 40 nm, such as the product sold under the trade name “T805” by the company Degussa Silices, TiO₂ treated with a polydimethylsiloxane and for which the mean size of the elementary particles is 21 nm, such as the product sold under the trade name “70250 Cardre UF TiO2SI3” by the company Cardre, anatase/rutile TiO₂ treated with a polydimethylhydrogenosiloxane and for which the mean size of the elementary particles is 25 nm, such as the product sold under the trade name “Microtitanium Dioxide USP Grade Hydrophobic” by the company Color Techniques.

The uncoated titanium oxide nanopigments are sold, for example, by the company Tayca under the trade names “Microtitanium Dioxide MT 500 B” or “Microtitanium Dioxide MT 600 B”, by the company Degussa under the name “P 25”, by the company Wackher under the name “Oxyde de titane transparent PW”, by the company Myoshi Kasei under the name “UFTR”, by the company Tomen under the name “ITS” and by the company Tioxide under the name “Tioveil AQ”.

The uncoated zinc oxide nanopigments are, for example: those sold under the name “Z-Cote” by the company Sunsmart; those sold under the name “Nanox” by the company Elementis; those sold under the name “Nanogard WCD 2025” by the company Nanophase Technologies.

The coated zinc oxide nanopigments are, for example: those sold under the name “Zinc Oxide CS-5” by the company Toshibi (ZnO coated with polymethylhydrogenosiloxane); those sold under the name “Nanogard Zinc Oxide FN” by the company Nanophase Technologies (as a 40% dispersion in Finsolv TN, C.sub.12-C.sub.15 alkyl benzoate); those sold under the name “Daitopersion ZN-30” and “Daitopersion ZN-50” by the company Daito (dispersions in cyclopolymethylsiloxane/oxyethylenated polydimethylsiloxane, containing 30% or 50% of nanozinc oxides coated with silica and polymethylhydrogenosiloxane); those sold under the name “NFD Ultrafine ZNO” by the company Daikin (ZnO coated with perfluoroalkyl phosphate and copolymer based on perfluoroalkylethyl as a dispersion in cyclopentasiloxane); those sold under the name “SPD-Z1” by the company Shin-Etsu (ZnO coated with silicone-grafted acrylic polymer, dispersed in cyclodimethylsiloxane); those sold under the name “Escalol Z100” by the company ISP (alumina-treated ZnO dispersed in an ethylhexyl methoxycinnamate/PVP-hexadecene/methicone copolymer mixture); those sold under the name “Fuji ZNO-SMS-10” by the company Fuji Pigment (ZnO coated with silica and polymethylsilsesquioxane); those sold under the name “Nanox Gel TN” by the company Elementis (ZnO dispersed at a concentration of 55% in C₁₂-C₁₅ alkyl benzoate with hydroxystearic acid polycondensate).

The uncoated cerium oxide nanopigments are sold under the name “Colloidal Cerium Oxide” by the company Rhone-Poulenc.

The uncoated iron oxide nanopigments are sold, for example, by the company Arnaud under the names “Nanogard WCD 2002 (FE 45B)”, “Nanogard Iron FE 45 EL AQ”, “Nanogard FE 45R AQ” and “Nanogard WCD 2006 (FE 45R)” or by the company Mitsubishi under the name “TY-220.”

The coated iron oxide nanopigments are sold, for example, by the company Arnaud under the names “Nanogard WOO 2008 (FE 455 FN)”, “Nanogard WCD 2009 (FE 45B 556)”, “Nanogard FE 45 BL 345” and “Nanogard FE 45 BL” or by the company BASF under the name “Transparent Iron Oxide”.

Mention may also be made of mixtures of metal oxides, especially of titanium dioxide and of cerium dioxide, including the silica-coated equal-weight mixture of titanium dioxide and of cerium dioxide, sold by the company Ikeda under the name “Sunveil A”, and also the alumina, silica and silicone-coated mixture of titanium dioxide and of zinc dioxide, such as the product “M 261” sold by the company Kemira, or the alumina, silica and glycerol-coated mixture of titanium dioxide and of zinc dioxide, such as the product “M 211” sold by the company Kemira.

The nanopigments may be introduced into the compositions according to the invention in unmodified form or in the form of pigmentary paste, i.e., as a mixture with a dispersant, as described, for example, in document GB-A-2 206 339.

Film Forming Polymers

Compositions of the present invention may include a film-forming polymer to enhance film formation and provide some water resistance. By “film-forming polymer,” it is meant a polymer that when dissolved in the composition, permits a continuous or semi-continuous film to be formed when the composition is spread onto, e.g., smooth glass, and the liquid vehicle is allowed to evaporate. As such, the polymer should dry on the glass in a manner in which over the area which it is spread should be predominantly continuous, rather than forming a plurality of discrete, island-like structures. Generally, the films formed by applying compositions on the skin according to embodiments of the invention described herein, are less than, on average, about 100 microns in thickness, such as less than about 50 microns.

Suitable film-forming polymers include natural polymers, such as polysaccharides or proteins and synthetic polymers, such as polyesters, polyacrylics, polyurethanes, vinyl polymers, polysulfonates, polyureas, polyoxazolines, and the like. Specific examples of film-forming polymers include, for example, acrylic homopolymers or copolymers with hydrophobic groups such as acrylate/ocylacrylamide copolymers including DERMACRYL 79 available from Akzo Chemical of Bridgewater, N.J.; dimethicone/acrylates dimethicone copolymer available as X-22-8247D from Shin-Etsu of Japan; hydrogenated dimer dilinoleyl/dimethylcarbonate copolymer, available from Cognis Corporation of Ambler, Pa. as COSMEDIA DC; copolymer of vinylpyrrolidone and a long-chain a-olefin, such as those commercially available from ISP Specialty Chemicals of Wayne, N.J. as GANEX V220; vinylpyrrolidone/tricontanyl copolymers available as GANEX WP660 also from ISP; water-dispersible polyesters, including sulfopolyesters such those commercially available from Eastman Chemical as EASTMAN AQ 38S. In certain embodiments, the film-forming polymer is water insoluble, but is rendered soluble upon exposure to alkalinity in order to facilitate removal from the skin upon washing with soap.

The amount of film-forming polymer present in the composition may be from about 0.25% to about 15%, or from about 0.5% to about 10%, or from about 1% to about 3%.

Particle Size

The size of the particles of the meta-stable composition in the form of an oil-in-water emulsion are about 0.2 microns (200 nm) to about 10 microns (10,000 nm) or alternatively about 0.2 microns (200 nm) to about 5 microns (5000 nm).

The meta-stable composition can be made utilizing any suitable mixing device, such as a rotor stator. However, in another embodiment, the meta-stable composition may be formed using a high pressure homogenizer. Formation using the high pressure homogenizers permit control of the particle size.

Hydrophilic-Lipophilic Balance (HLB)

Hydrophile-lipophile balance is an empirical expression for the relationship of the hydrophilic (“water-loving”) and hydrophobic (“water-hating”) groups of a surfactant. The HLB system is particularly useful to prepare O/W and W/O emulsions. Water-in-oil emulsions (W/O) require low HLB surfactants, having an HLB value of equal to or less than 6. Oil-in-water emulsions (O/W) require high HLB surfactants having an HLB value equal to or greater than 9. The total HLB of surfactants can be calculated by taking the weight percent of each surfactant of the surfactant mixture and multiplying the weight percent by the HLB to obtain a contribution of each individual surfactant on the HLB. The individual contributions are then added together to get the total HLB of the surfactant mixture. HLBs for individual surfactants are usually provided in the literature or provided by the surfactant supplier. For example, if the surfactant mixture is a 50/30/20 blend of polysorbate 61, octyldodecyl xyloside and glyceryl stearate the total HLB of the 50/30/20 surfactant mixture is 7.11. The HLB of polysorbate 61 is 9.4, the HLB of octyldodecyl xyloside is 5.5, and the HLB of glyceryl stearate 3.8. The contribution of polysorbate 61 is 0.5×9.4=4.7. The contribution of octyldodecyl xyloside is 0.3×5.5=1.65. The contribution of glyceryl stearate is 0.2×3.8=0.76. The total HLB is 4.7+1.65+0.76=7.11.

The meta-stable photoprotective composition has a ratio of the at least one non-ionic oil-in-water emulsifier to the at least one water-in-oil emulsifier is about 0.2 to about 2.0, or alternatively about 0.3 to about 1.0, or alternatively about 0.3 to about 0.5.

Viscosity

Viscosity is measured using Brookfield Viscometer, in centipoise (mPa·s) using spindle T-D with speed set at 100 rpm. In one embodiment, the viscosity of the meta-stable photoprotective composition is less than 400 centipoise (cps). In another embodiment, the viscosity of the composition is less than about 150 centipoise (cps).

A method for treating keratinous tissue includes applying to the keratinous tissue the composition of the present disclosure. The meta-stable photoprotective composition of the present disclosure is in any desirable cosmetic form, such as, but not limited to, sprays, liquid lotions, creams, mousses, and foams, can be applied to keratinous tissue to provide the desired cosmetic effect, such as, but not limited to, UV protection or hydration.

The compositions described above may be packaged, in a known manner, in a single-compartment bottle. The user must then shake the bottle before pouring or spraying the product onto skin. The product may also be packed in a bottle of the “pump dispenser” type. Provision may also be made for the two phases of the composition to be introduced into two independent compartments of the same bottle, a system being provided to mix them together at the time of dispensing. Such devices are described, for example, in documents EP-A-497 256 and FR-A-2 697 233. In another embodiment, the product may also be packed in an aerosol packaging system, such as a bag in can, bag on valve, piston in can or other similar packaging having two or more separate compartments or storage areas for the product and propellant.

Processes that can be used to make the inventive compositions include, but are not limited to, classical mixing, high pressure homogenization, Symex, hold-cold, or dilution.

The present invention will be better understood from the examples that follow, all of which are intended for illustrative purposes only and are not meant to unduly limit the scope of the invention in any way.

### EXAMPLES

Having generally described this invention, a further understanding can be obtained by reference to certain specific examples which are provided herein for purposes of illustration only and are not intended to be limiting unless otherwise specified. The amounts shown are in % by weight, unless otherwise mentioned.

Inventive Composition

TABLE 1

| | Chemical name | Ex. 1 |
|---|---|---|
| Phase A | Water | Q.S. |
| | Ethylenediaminetetraacetic acid (EDTA) | 0.1 |
| | Preservative | 0.8 |
| | Polysorbate-60 | 0.5 |
| | Sodium Stearoyl Glutamate | 0.5 |
| Phase B | Avobenzone | 2.2 |
| | Octocrylene | 4.5 |
| | Benzophenone-3 | 2.9 |
| | Octisalate | 2.4 |
| | Homosalate | 8.0 |
| | Emollient | 6.7 |
| | Octyldodecanol (and) Octyldodecyl Xyloside | 1.1 |
| | Glyceryl Stearate | 0.4 |
| | Stearyl Alcohol | 0.2 |
| | Preservative | 0.1 |
| | Antioxidant | 0.1 |
| | Aesthetic modifier | 4.3 |
| Phase C | SPF Booster | 4.0 |
| Phase D | Wetting agent | 0.3 |
| | Total (% wt/wt) | 100.0 |
| Properties | Viscosity (centipoise) | 164 |
| | Weight Ratio of W/O Emulsifier to O/W Emulsifier | 0.31 |
| | In-Vivo Static SPF | 49 |
| | 80 Minute Water Resistant SPF | 48 |
| | Water Resistance (Yes/No) | Yes |
| | Inversion Confirmed | Yes |

In making each of the examples in Table 1, the following procedure is used. Phase A ingredients are weighed and added to a main reactor and heated to about 90° C. Phase B ingredients are weighed and added to a second reactor and heated to 90° C. Phase B ingredients are added to the main reactor containing Phase A ingredients and homogenized for 30 minutes while the temperature is maintained at 90° C. Next the mixture is removed from heat and allowed to begin to cool to room temperature. A high pressure homogenizer is used to make 2 runs at 500 bar. Phase C is then added into the main reactor. The mixture is homogenized for 20 to 30 minutes. Next, the reaction mixture is cooled to about 25° C. and Phase D is added to the main reactor and stirred. The mixture is cooled to room temperature. The viscosity of the mixture is 164 centipoise, which is measured using a Brookfield Viscometer, using spindle T-D and speed set at 100 rpm. The inventive composition of Table 1, Example 1, is a meta-stable composition that is applied as an oil-in-water (O/W) emulsion which quickly inverts to a water-in-oil emulsion (W/O) upon rubbing into the skin and that is pleasing to consumers.

The composition of Table 1 was further tested to confirm inversion of the phases from an O/W emulsion to a W/O emulsion for varying concentrations of water. The point of inversion for the inventive composition occurs at a weight ratio of oil phase to water phase of between about 1.4 to about 2.3 or about 60/40 to about 70/30.

Other processes that can be used to make the inventive compositions include, but are not limited to, classical mixing, high pressure homogenization, Symex, hold-cold, or dilution.

TABLE 2

| | | | Comparative | Ex. 2 | Ex. 3 | Ex. 4 | Ex. 5 | Ex. 6 |
|---|---|---|---|---|---|---|---|---|
| Phase | Chemical Name | HLB | % wt/wt | % wt/wt | % wt/wt | % wt/wt | % wt/wt | % wt/wt |
| A-1 | Water | | | 29.1 | 29.1 | 29.1 | 29.1 | 29.1 |
| | PEG-40 Stearate | 16.9 | | 0.5 | 0.5 | 0.5 | 0.5 | 0.5 |
| | Polysorbate-61 | 9.4 | | | | | | |
| A-2 | Water | | 59.2 | 30.0 | 30.0 | 30.0 | 30.0 | 30.0 |
| | EDTA | | 0.1 | 0.1 | 0.1 | 0.1 | 0.1 | 0.1 |
| | Preservative | | 0.5 | 0.5 | 0.5 | 0.5 | 0.5 | 0.5 |
| | Sodium Stearoyl Glutamate | | 0.8 | 0.5 | 0.5 | 0.5 | | |
| | Potasium cetyl phosphate | | | | | | 0.5 | 0.5 |
| | Disodium Steoryl Glutamate | | | | | | | |
| B | Avobenzone | | 3.0 | 3.0 | 3.0 | 3.0 | 3.0 | 3.0 |
| | Octocrylene | | 7.0 | 7.0 | 7.0 | 7.0 | 7.0 | 7.0 |
| | Benzophenone-3 | | 6.0 | 6.0 | 6.0 | 6.0 | 6.0 | 6.0 |
| | Octyl Salicylate | | 5.0 | 5.0 | 5.0 | 5.0 | 5.0 | 5.0 |
| | Homosalate | | 10.0 | 10.0 | 10.0 | 10.0 | 10.0 | 10.0 |
| | Sucrose Distearate | 3.0 | | 1.2 | | | | |
| | Polyglyceryl-4 Isostearate (and) Cetyl | 5.0 | | | 1.2 | | | 1.2 |
| | PEG-PPG 10/1 Dimethicone (and) | | | | | | | |
| | Hexyl Laurate | | | | | | | |
| | Sorbitan Stearate | 4.7 | | | | 1.2 | 1.2 | |
| | Octyldodecanol | 5.5 | 1.6 | | | | | |
| | (and)Octyldodecylxyloside | | | | | | | |
| | Glyceryl Stearate | 3.8 | 0.3 | 0.4 | 0.4 | 0.4 | 0.4 | 0.4 |
| | Stearyl Alcohol | | | 0.2 | 0.2 | 0.2 | 0.2 | 0.2 |
| | Preservative | | 0.4 | 0.4 | 0.4 | 0.4 | 0.4 | 0.4 |
| | Aesthetic modifier | | 5.5 | 5.5 | 5.5 | 5.5 | 5.5 | 5.5 |
| | Vitamin E | | 0.1 | 0.1 | 0.1 | 0.1 | 0.1 | 0.1 |
| | Wetting agent | | 0.5 | 0.5 | 0.5 | 0.5 | 0.5 | 0.5 |

| | | | | | | | |
|---|---|---|---|---|---|---|---|
| Properties | Weight Ratio of O/W Emulsifier to W/O | 0.42 | 0.31 | 0.31 | 0.31 | 0.31 | 0.31 |
| | Emulsifier | | | | | | |

| | | | Ex. 7 | Ex. 8 | Ex. 9 | Ex. 10 | Ex. 11 | Ex. 12 |
|---|---|---|---|---|---|---|---|---|
| Phase | Chemical Name | HLB | % wt/wt | % wt/wt | % wt/wt | % wt/wt | % wt/wt | % wt/wt |
| A-1 | Water | | 29.1 | 29.1 | 29.1 | 29.1 | 29.1 | 29.1 |
| | PEG-40 Stearate | 16.9 | 0.5 | 0.5 | 0.5 | 0.5 | 0.5 | |
| | Polysorbate-61 | 9.4 | | | | | | 0.5 |
| A-2 | Water | | 30.0 | 30.0 | 30.0 | 30.0 | 30.0 | 30.0 |
| | EDTA | | 0.1 | 0.1 | 0.1 | 0.1 | 0.1 | 0.1 |
| | Preservative | | 0.5 | 0.5 | 0.5 | 0.5 | 0.5 | 0.5 |
| | Sodium Stearoyl Glutamate | | | | | | | 0.5 |
| | Potasium cetyl phosphate | | 0.5 | | | | | |
| | Disodium Steoryl Glutamate | | | 0.5 | 0.5 | 0.5 | 0.5 | |
| B | Avobenzone | | 3.0 | 3.0 | 3.0 | 3.0 | 3.0 | 3.0 |
| | Octocrylene | | 7.0 | 7.0 | 7.0 | 7.0 | 7.0 | 7.0 |
| | Benzophenone-3 | | 6.0 | 6.0 | 6.0 | 6.0 | 6.0 | 6.0 |
| | Octyl Salicylate | | 5.0 | 5.0 | 5.0 | 5.0 | 5.0 | 5.0 |
| | Homosalate | | 10.0 | 10.0 | 10.0 | 10.0 | 10.0 | 10.0 |
| | Sucrose Distearate | 3.0 | 1.2 | 1.2 | | | | |
| | Polyglyceryl-4 Isostearate (and) | 5.0 | | | 1.2 | | | |
| | Cetyl PEG-PPG 10/1 | | | | | | | |
| | Dimethicone (and) Hexyl Laurate | | | | | | | |
| | Sorbitan Stearate | 4.7 | | | | 1.2 | | |
| | Octyldodecanol | 5.5 | | | | | 1.2 | 1.2 |
| | (and)Octyldodecylxyloside | | | | | | | |
| | Glyceryl Stearate | 3.8 | 0.4 | 0.4 | 0.4 | 0.4 | 0.4 | 0.4 |
| | Stearyl Alcohol | | 0.2 | 0.2 | 0.2 | 0.2 | 0.2 | 0.2 |
| | Preservative | | 0.4 | 0.4 | 0.4 | 0.4 | 0.4 | 0.4 |
| | Aesthetic modifier | | 5.5 | 5.5 | 5.5 | 5.5 | 5.5 | 5.5 |
| | Vitamin E | | 0.1 | 0.1 | 0.1 | 0.1 | 0.1 | 0.1 |
| | Wetting agent | | 0.5 | 0.5 | 0.5 | 0.5 | 0.5 | 0.5 |

| | | | | | | | |
|---|---|---|---|---|---|---|---|
| Properties | Weight Ratio of O/W Emulsifier to W/O | 0.31 | 0.31 | 0.31 | 0.31 | 0.31 | 0.31 |
| | Emulsifier | | | | | | |

The Comparative Example provided in Table 1 is a sunscreen composition that does not include a nonionic O/W emulsifier. The Comparative Example forms O/W emulsion and it doesn't invert into W/O emulsion and does not provide water resistance when applied to keratinous tissues.

Sensory Test

To evaluate sensory skin perception, the composition according to Example 12 (SPF50+) but in a sprayable form and two commercially available sprayable sunscreens (Sublime Continuous Clear Spray SPF 50+ and Sublime Sun Lotion Spray SPF50+) were tested on 12 men and women who preferably use a spray sunscreen with a SPF15 or higher. The subjects were presented with the test products in blinded, aerosol packaging and did at home testing. Each of the 12 men and women applied each of the sunscreens to the face and body and provided opinions, satisfaction, and overall preference to the tested products. The aspects that the 12 men and women were requested to rate were texture, application, efficacy, fragrance and overall satisfaction. The following is a summary of the results:

TABLE 4
Sensory Test Findings

| | | Sublime | |
|---|---|---|---|
| Subjects' Sensory Comments | | Continuous | Sublime Sun |
| (All to Most) | Example 12 | Clear Spray | Lotion Spray |
| Appropriate color | Yes | Yes | Yes |
| Even spray distribution | Yes | Yes | No |
| Pleasant Texture | Yes | Yes | No |
| Smooth Texture | Yes | Yes | No |
| Easy Application | No | Yes | No |
| Greasy to non/greasy texture | Yes | No | No |
| No residue | Yes | No | No |
| Light Feel on Skin | No | Yes | No |
| No noodling | Yes | Yes | Yes |
| Greasy to non/greasy skin feel | Yes | No | No |
| Sticky to non-sticky skin feel | Yes | No | Yes |
| Smooth Skin feel | Yes | Yes | Yes |
| Adequate moisture | Yes | No | No |
| Protected Skin Feel | Yes | No | Yes |
| Amount of shine on skin | Yes | No | No |
| Comfortable skin feel | Yes | Yes | No |
| No irritation | Yes | No | No |
| Overall satisfaction | Yes | N/A | N/A |

Overall 10 out of 12 subjects gave above average ratings and were somewhat/very satisfied with the sprayable sunscreen composition like Example 12. The subjects specifically mentioned the non-greasy skin feel of sprayable sunscreen composition like Example 12. Both the Sublime Clear Continuous Clear Spray and the Sublime Sun Lotion Spray received below satisfaction ratings with only 6 of the 12 subjects being somewhat/very satisfied, and the subjects specifically mentioned that the skin felt too greasy with both of the sunscreens.

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

1. A meta-stable photoprotective composition comprising:
at least one water-in-oil emulsifier having a hydrophilic-lipophilic balance (HLB) of less than 6;
at least one non-ionic oil-in-water emulsifier having an HLB of greater than 9;
at least one ionic surfactant; and
at least one sunscreen active ingredient;
wherein the weight ratio of the at least one non-ionic oil-in-water emulsifier to the at least one water-in-oil emulsifier is about 0.2 to about 2.0, and
wherein the composition is an oil-in-water emulsion that inverts to a water-in-oil emulsion upon rubbing.

2. The meta-stable photoprotective composition of claim 1, wherein the combined concentration by weight of the at least one water-in-oil emulsifier, the at least one non-ionic oil-in-water emulsifier and the at least one ionic surfactant does not exceed about 4% by weight, based on the total weight of the composition.

3. The meta-stable photoprotective composition of claim 1, wherein the viscosity of the composition is less than about 400 centipoise (cps).

4. The meta-stable photoprotective composition of claim 1, wherein the viscosity of the composition is less than about 150 centipoise (cps).

5. The meta-stable photoprotective composition of claim 1, wherein the at least one water-in-oil emulsifier is present in an amount of from about 0.7% to about 3.0% by weight, based on the total weight of the composition.

6. The meta-stable photoprotective composition of claim 1, wherein the at least one non-ionic oil-in-water emulsifier is present in an amount of from about 0.2% to about 2.0% by weight, based on the total weight of the composition.

7. The meta-stable photoprotective composition of claim 1, wherein the at least one ionic surfactant is present in an amount of from about 0.1% to about 1.0% by weight, based on the total weight of the composition.

8. The meta-stable photoprotective composition of claim 1, wherein the at least one sunscreen active ingredient is present in an amount of from about 1% to about 40% by weight, based on the total weight of the composition.

9. The meta-stable photoprotective composition of claim 1, wherein the at least one water-in-oil emulsifier is sucrose distearate, polyglyceryl-4 isostearate (and) cetyl PEG/PPG 10/1 dimethicone (and) hexyl laurate, sorbitan stearate, octyldodecyl xyloside, glyceryl stearate, and combinations thereof.

10. The meta-stable photoprotective composition of claim 1, wherein the at least one non-ionic oil-in-water emulsifier is chosen from PEG-40 stearate, polysorbate-61, and combinations thereof.

11. The meta-stable photoprotective composition of claim 1, wherein the at least one ionic surfactant is chosen from sodium stearoyl glutamate, potassium cetyl phosphate, disodium stearoyl glutamate, and combinations thereof.

12. The meta-stable photoprotective composition of claim 1, wherein the sunscreen active ingredient is a sunscreen active.

13. (canceled)

14. The meta-stable photoprotective composition of claim 1, wherein the size of the particles of the oil-in-water emulsion are about 0.2 microns (200 nm) to about 10 microns (10,000 nm).

15. The meta-stable photoprotective composition of claim 1, wherein the size of the particles of the oil-in-water emulsion are about 0.2 microns (200 nm) to about 5 microns (5000 nm).

16. The meta-stable photoprotective composition of claim 1, wherein the composition includes an oil phase, the oil phase containing surfactants, wherein the surfactants comprise less than 15% by weight of the oil phase.

17. The meta-stable photoprotective composition of claim 1, wherein the composition includes an oil phase, the oil phase containing surfactants, wherein the surfactants comprise less than 10% by weight of the oil phase.

18. The meta-stable photoprotective composition of claim 1, wherein and the composition is water-resistant upon rubbing the composition into the skin

19. A product formed from a meta-stable photoprotective composition comprising:
at least one water-in-oil emulsifier having a hydrophilic-lipophilic balance (HLB) of less than 6;
at least one non-ionic oil-in-water emulsifier having an HLB of greater than 9;
at least one ionic surfactant; and
at least one sunscreen active ingredient;
wherein the ratio of the at least one non-ionic oil-in-water emulsifier to the at least one water-in-oil emulsifier is about 0.2 to about 2.0 and
wherein the composition is an oil-in-water emulsion that inverts to a water-in-oil emulsion upon rubbing.
